(19) United States
(12) Patent Application Publication
(10) Pub. No.: US 2014/0242587 A1
(43) Pub. Date: Aug. 28, 2014

(54) Rapid and Reliable Detection of Infectious Agents

(71) Applicant: University of Florida Research Foundation, Inc., Gainesville, FL (US)

(72) Inventors: Kenneth H. Rand, Gainesville, FL (US); Herbert J. Houck, Gainesville, FL (US)

(21) Appl. No.: 14/349,506

(22) PCT Filed: Oct. 3, 2012
(86) PCT No.: PCT/US12/00489

§ 371 Date: Apr. 3, 2014

Related U.S. Application Data
(60) Provisional application No. 61/542,470, filed on Oct. 3, 2011.
(60) Provisional application No. 61/550,424, filed on Oct. 23, 2011.
(60) Provisional application No. 61/655,071, filed on Jun. 4, 2012.

(51) Int. Cl. C12Q 1/68; C12N 9/12

(52) U.S. Cl. CPC C12Q 1/689; C12N 9/1276
USPC 435/6.11; 536/24.33; 536/24.32; 435/287.2; 435/194

(57) ABSTRACT

The present invention is directed to devices, systems and methods that enable the detection of low copy numbers of bacterial polynucleotides in a sample without having to use multiple species specific primer sequences.

### CROSS-REFERENCE TO RELATED APPLICATIONS

This application is related to U.S. Provisional Application No. 61/542,470 filed Oct. 3, 2011, U.S. Provisional Application No. 61/550,424 filed Oct. 23, 2011, and U.S. Provisional Application No. 61/655,071 filed Jun. 4, 2012 to which priority is claimed under 35 USC 119 and which are incorporated herein by reference in their entirety.

### FIELD OF THE INVENTION

The present invention relates to a device, system and apparatus for detecting bacterial infections in biological materials.

### BACKGROUND OF THE INVENTION

Development of a rapid diagnostic test for detecting bacterial infection would have a significant impact on the management of infections. For the identification of pathogens and antibiotic resistance genes in clinical samples, DNA probe and DNA amplification technologies offer several advantages over conventional methods. The organism can be detected directly in clinical samples, thereby reducing the cost and time associated with isolation of pathogens. Also, bacterial genotypes (at the DNA level) are more stable than the bacterial phenotypes (i.e. biochemical properties). DNA-based technologies have proven to be extremely useful for specific applications in the clinical microbiology laboratory (and a method to quantify small amounts of DNA). For example, kits for the detection of fastidious organisms based on the use of hybridization probes or DNA amplification for the direct detection of pathogens in clinical specimens are commercially available (Persing et al, 1993. Diagnostic Molecular Microbiology: Principles and Applications, American Society for Microbiology, Washington, D.C.).

The conventional DNA-based tests for the detection and identification are based on the amplification of the highly conserved 16S rRNA gene followed by hybridization with internal species-specific oligonucleotides. The significance of the 16SrRNA gene is that certain sequences are conserved in all gene variants. The subsequent hybridization targets and allows for amplification of species-specific oligonucleotides which are derived from species-specific bacterial genomic DNA fragments. However, ultimately, these conventional strategies using universal sequences suffer from the fact that the use of Taq polymerase interferes with the detection. Contamination of the Taq polymerase with bacterial nucleic acid was first described over 20 years ago. See Rand and Houck, Molecular and Cellular Probes (1990) 4:445-450. This means if one uses primers targeting areas of the 16 S ribosomal RNA (or DNA) that are shared by many bacteria, the contamination of the Taq becomes a limiting factor in detecting low copy numbers of bacteria. In applying such a method to the detection of bacteria in normally sterile clinical specimens, the Taq enzyme contamination forces the use of primers specific to various species of bacteria, rather than allowing the use of sequences that could amplify all or many species.

### BRIEF DESCRIPTION OF THE DRAWINGS

FIGS. 1a-1e show a stepwise diagram of a probe embodiment and method of using the probe to selectively amplify a target product.

FIG. 2 shows a gel that demonstrates how specific and sensitive the method embodiment is at capturing and detecting bacterial nucleic acid material in a sample.

FIG. 3 shows the temperature dependence of the false positive product with reagents alone i.e. that if the Tm of the universal part of the fusion primer is low enough there can be no PCR product if the PCR is carried out at a high enough temperature.

FIG. 4a-4b show proof of principle that includes both dilution of the RT reaction mixture (1:50) and by using a high enough annealing temperature in the PCR (68° C. in FIG. 4; 65° C. in FIG. 5).

FIGS. 5a-5f show a stepwise diagram of a probe embodiment and method of using the probe to selectively amplify a target product.

### DETAILED DESCRIPTION OF THE INVENTION

Aspects of the present invention are directed to devices, systems and methods that enable the detection of low copy numbers of bacterial polynucleotides in a sample without having to use multiple species specific primer sequences. In this way, aspects of the present invention provide highly sensitive diagnostic tests capable of detecting essentially all potential bacterial pathogens in a biological sample within a short period of time, e.g., hours. In one aspect, an initial primer is utilized comprising a non-bacterial sequence interconnected to a universal bacterial sequence. The universal sequence is removed, destroyed, inactivated, etc. such that it does not interfere in a PCR step by inhibiting the PCR itself and/or by causing a false positive from the contaminating bacterial DNA in the Taq enzyme.

In accordance with one aspect, the present invention pertains to a probe for detecting target nucleic acid material in a sample. The target nucleic acid material may comprise polynucleotides from any organism or virus, including but not limited to plant and animal polynucleotides. In one embodiment, the target nucleic acid is a bacterial, fungal, viral, or other infectious agent. The probe may contain a universal probe sequence hybridizable to a target sequence. There is interconnected to the probe sequence, whether adjacent or non-adjacent, a unique primer sequence.

The unique primer sequence is engineered to have an arbitrary sequence that hybridizes to a unique primer. Thus, the unique primer sequence may be utilized to develop a primer for use in an amplification step as will be explained in further detail below. In addition, the arbitrary sequence is one that avoids undesired binding with the target sequence or possible nucleic acid contaminants in the sample. Contaminants would be nucleic acid sequences in the test reagents that if amplified would interfere with detection of the target nucleic acid in a patient (or other) sample of interest, e.g., including nucleic acids from any organism whether bacterial, fungal, other infectious agent or even human, animal and plant. The probe sequence and unique primer sequence are typically on the same strand and, in certain embodiments, are associated with a solid phase medium.

In another aspect, the probe includes a universal probe sequence hybridizable with polynucleotide sequences of multiple bacterial species and a non-bacterial primer sequence interconnected with the universal probe sequence. In one embodiment, the probe may further comprise a solid-phase medium associated with the universal probe sequence and the non-bacterial primer sequence; alternatively the solid-phase medium may be associated with the 2^{nd} universal primer used in the PCR with the unique primer. In certain embodiments, the universal probe sequence comprises a DNA sequence or an RNA sequence. The universal probe sequence and the non-bacterial primer sequence may be on the same strand. In one embodiment, the non-bacterial primer sequence includes a sequence of at least 5, 10, 15, 20, or 25 bases that are lacking in 10 or more natural species of bacteria.

When utilized, the solid-phase medium may be any suitable medium for binding of the universal probe so that the probe is isolated to enhance sensitivity and yield downstream. In one embodiment, the solid-phase medium comprises a bead. In a particular embodiment, the bead comprises a magnetic bead. In alternative embodiments, the solid-phase medium comprises wall of a well, dish or other container capable of holding a fluid. The probe may further include an adenine strand linked to the non-bacterial primer sequence on one end and to Biotin on the other end. In this embodiment, the Biotin is typically bound to the bead.

In further embodiments, the universal probe sequence is an RNA or DNA sequence specific to 16S RNA of multiple bacterial species. In one embodiment, the universal probe sequence is used to target a region of 16SrRNA and to amplify the target in parts. In a particular embodiment, the universal probe sequence is engineered to bind to >90% of known bacterial isolates.

According to another aspect, the invention pertains to a method of detecting bacterial nucleic acid material in a sample. The method includes contacting the sample with a probe comprising: (i) a universal probe sequence hybridizable with polynucleotide sequences of multiple bacterial species; and (ii) a non-bacterial primer sequence interconnected with the universal probe sequence. The method further comprises selectively amplifying any bacterial nucleic acid material in the sample that is captured by the probe. In one embodiment, the bacterial nucleic acid material captured by the probe comprises a DNA or an RNA sequence. In certain embodiments, the probe further comprises (iii) a solid-phase medium associated with the universal probe sequence and the non-bacterial primer sequence.

In accordance with another aspect, the captured nucleic acid is an RNA sequence and the method further comprises step of subjecting the RNA sequence to reverse transcriptase under conditions to produce a DNA extension on the same strand as the universal probe sequence, the DNA extension being complementary to a portion of the RNA sequence not hybridized to the universal probe sequence. The universal probe sequence and DNA extension may form a base strand. This strand may be made double-stranded in one embodiment by enzymatic methods as are well-known in the art. The method may further comprise a selectively amplifying step, which may be a polymerase chain reaction (PCR) using the base strand. The use of PCR may include the implementation of real-time PCR. Further, the PCR may include combining the base strand, whether associated with said solid-phase medium or not, in a reaction mixture with a first primer complimentary to the non-bacterial primer sequence and a second primer complimentary to a sequence on the DNA extension.

In accordance with another aspect, there is provided a method of detecting target nucleic acid material in a sample. The method comprises contacting the sample with an initial probe comprising: (a) a universal probe sequence as described herein hybridizable with polynucleotide sequences of multiple bacterial species; and (b) a non-bacterial sequence interconnected with the universal probe sequence. The method comprises subjecting a captured polynucleotide sequence to reverse transcriptase under conditions to produce a base strand comprising a DNA extension on the same strand as the universal probe sequence. In one embodiment, the DNA extension is complementary to a portion of the RNA sequence not hybridized to the universal probe sequence. This strand may be made double-stranded in one embodiment by enzymatic methods as are well-known in the art.

Thereafter, the method comprises conducting a polymerase chain reaction (PCR) using a base strand comprising the non-bacterial sequence, the universal probe sequence and the DNA extension. The PCR comprises: with the target DNA dissolved in a PCR reaction mixture comprising the base strand, primers, a DNA polymerase, heating said PCR reaction mixture sufficiently to achieve denaturation of the base strand into single-strand DNA. In this step, the primers comprise a first primer complimentary to the non-bacterial primer sequence and a second primer complimentary to a sequence on the DNA extension. In addition, the PCR comprises cooling the PCR reaction mixture sufficiently to cause the primers to anneal to the single-strand DNA and to elongate and thereby at least partially form a DNA strand complementary to the single-strand DNA. Further, the PCR comprises step (iii) of subjecting the PCR reaction mixture to a reaction temperature of about 65° C. to further elongate the complementary DNA strand formed in step (ii). Thereafter, the PCR steps may be repeated as is known in the art as desired.

Critically, the universal probe sequence interconnected to the universal primer sequence is rendered at least partially inoperable to participate in the PCR before the subsequent PCR step to avoid inhibition of the PCR or production of a false positive PCR product. For example, the universal probe sequence may be removed, destroyed, inactivated, diluted, or otherwise rendered non-functional etc. such that it does not interfere in a PCR step by inhibiting the PCR itself and/or by causing a false positive from the contaminating bacterial DNA in the Taq enzyme. It is appreciated therefore that the initial primer may be rendered inoperable by various methods as would be appreciated by one skilled in the art. In one embodiment, as explained below in the Examples below, the universal probe sequence is constructed so as to have a relatively low Tₘ. In one embodiment, the universal probe sequence has a Tₘ of from 45-55° C. Similarly, the non-bacterial sequence has a relatively high Tₘ. In one embodiment, the non-bacterial sequence has a sufficiently high Tₘ to allow for annealing in PCR at a temperature that is at least 10° C. greater than the universal probe sequence Tₘ. In one embodiment, the annealing temperature in the PCR is from 65-70° C. rather than the standard 55-60° C. Advantageously, because the Tₘ of the universal portion is so low, it can never hybridize with the contaminating DNA in the Taq enzyme during the PCR because the lowest temperature in the PCR remains 10° C. greater than the universal probe sequence Tₘ.

In one embodiment, the universal probe sequence is removed from the PCR reaction mixture so that it cannot participate in the subsequent PCR step. In this instance, the universal probe is removed by enzymatic digestion, or by physico-chemical means, or even sufficiently diluted. In another embodiment, the universal probe sequence is constructed in such a manner that it cannot participate in the subsequent PCR step. For example, the universal probe sequence may be shortened to the extent that it cannot form a PCR product in a subsequent PCR step. In another embodiment, the universal sequence is modified or contains modified nucleotides such that it cannot form a PCR product in the subsequent PCR step. In one embodiment, the modified nucleotides may be effective to increase the affinity of the universal sequence for RNA.

In another aspect, embodiments of the PCR method described herein further comprise diluting the PCR reaction mixture prior to the heating step of the PCR. In one embodiment, the PCR reaction mixture is diluted with a suitable medium, e.g., buffer, in a range of 1:20 to 1:60 by volume. Without wishing to be bound by theory, it is believed that the dilution of the PCR reaction mixture aids in increasing the signal to noise ratio in a subsequent detection step following PCR.

Detection of the amplified sequences may be accomplished utilizing well-known methods and devices in the art. Without limitation, detection may be accomplished by agarose gel and/or polyacrylamide gel electrophoresis, restriction endonuclease digestion, Dot blots, high pressure liquid chromatography (HPLC), electrochemilluminescence, and/or direct sequencing. Optionally, a suitable visualization technique may be utilized in combination therewith, such as by EtBr staining, Southern blotting, labeling, silver staining, hybridization with a labeled probe, UV detection, voltage-initiated chemical reaction photon detection, and/or radioactive or fluorescent-based DNA sequencing.

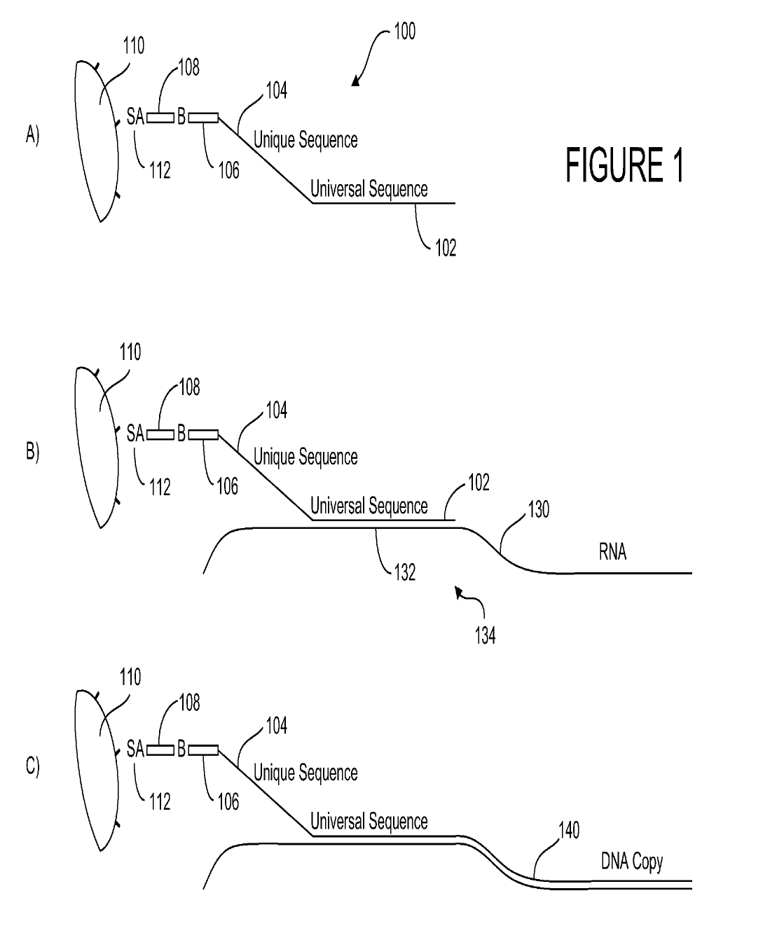

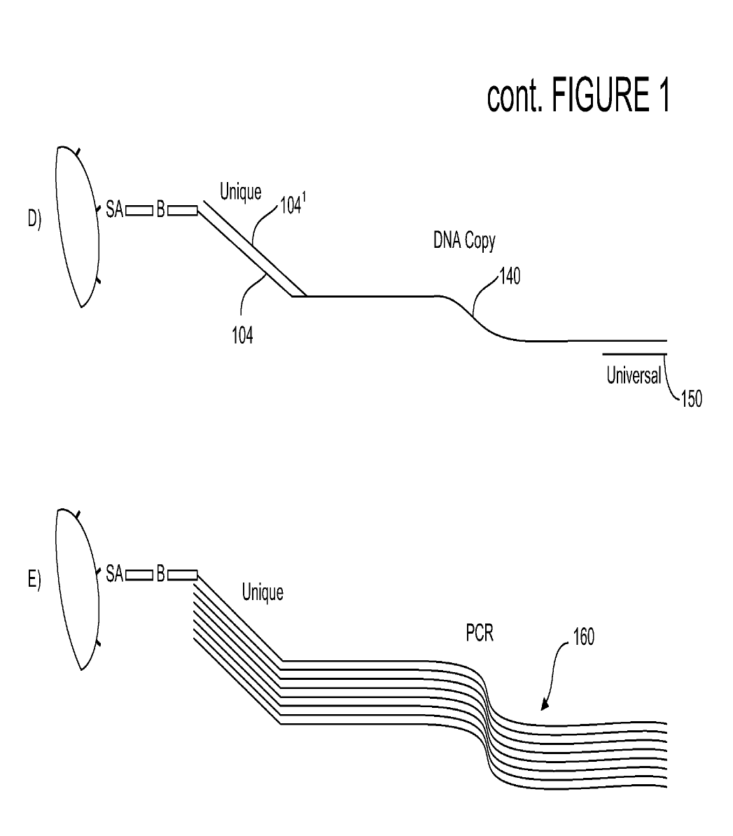

Turning now to the drawings, FIG. 1 depicts a stepwise method showing how bacterial nucleic acid material can be selectively captured and amplified, which in turn enables the identification of bacterial infection. This identification can occur even when there is a low copy number in the sample. FIG. 1 a shows a probe 100 that includes a specific probe sequence 102 that is universal to several bacterial species; thus, it may also be referred to as a “universal probe sequence” or “universal sequence.” The probe 100 also includes a unique sequence 104 on the same strand as the universal probe sequence 102. The unique sequence 104 is specifically designed to lack bacterial sequences, and typically pertains to at least 5, 10, 15, 20, or 25 bases. The unique sequence 104 may also be referred to as a non-bacterial sequence. The non-bacterial sequence 104 typically lacks homology or does not recognize bacterial sequences from at least 10 or more bacterial species.

The probe 100 further includes a linker sequence 106 adjacent to the non-bacterial sequence 104. The linker sequence 106 links to a biotin molecule 108. In one embodiment, the linker sequence 106 may comprise a series of adenine bases. The biotin 108 binds to a streptoavidin molecule 112 bound to a solid phase substrate 110. The solid phase substrate 110 shown is a magnetic bead, but it is understood that the present invention is not so limited. In operation, the probe 100 is exposed to a sample, such as, but not limited to, a biological fluid (blood, mucous, vaginal fluid, serum, semen etc.), tissue sample (typically a tissue sample expected of being infected, and may be homogenized), food sample, or any liquid or other sample (including nucleic acid extracts thereof) suspected of being infected with a bacterium. Typically, the sample is suspected to contain both human and bacterial RNA. RNA 130 in the sample hybridizes to the universal probe sequence 104 at a complimentary sequence 132 (FIG. 1 b). By isolating the bead 110, the captured RNA 130 is washed of non-bound nucleic acid.

The resulting DNA-RNA hybrid 134 attached to the bead 110 is used as the primer/template for reverse transcriptase (RT) to copy the hybridized bacterial RNA thereby forming a cDNA extension strand 140 (FIG. 1c). The bead 110 is then washed again. Using a PCR primer 150 likely to bind to a site downstream on the cDNA extension strand and a primer 104′ directed to the non-bacterial sequence 104 (FIG. 1d), PCR is further conducted (FIG. 1e) to amplify the target product to produce product 160. Even though universal bacterial sequences were used to capture the RNA and a bacterial universal primer was used as the PCR primer 150, the non-bacterial sequence 104 allows for completely specific amplification of the RNA that has been captured and copied. It is noted that regular PCR or real-time PCR (rtPCR), can be conducted to amplify the target sequences. rtPCR provides a more rapid means of detecting the presence of bacterial nucleic acid material in a sample.

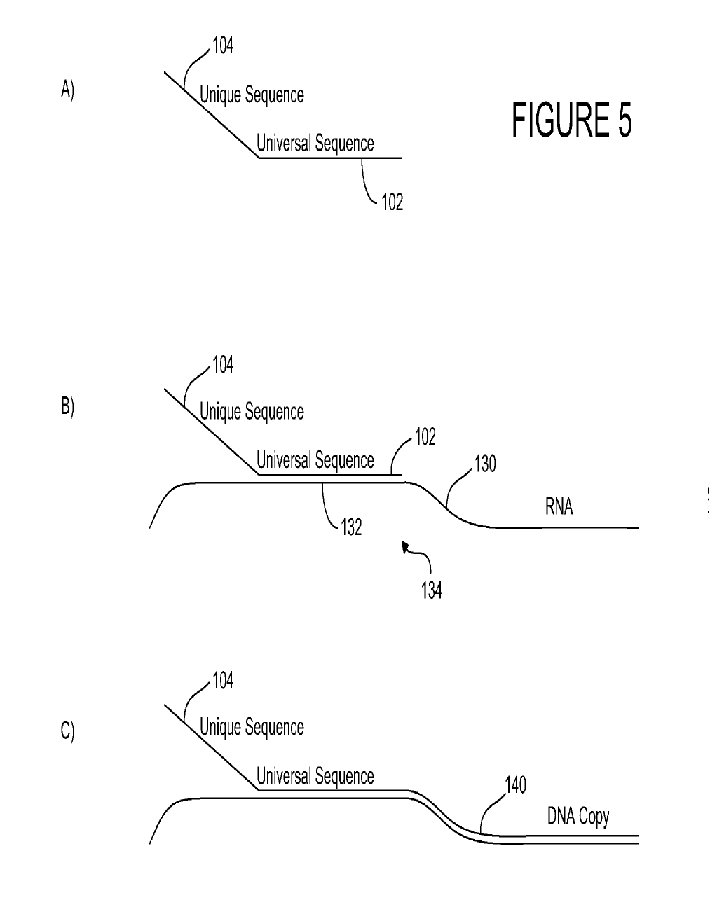

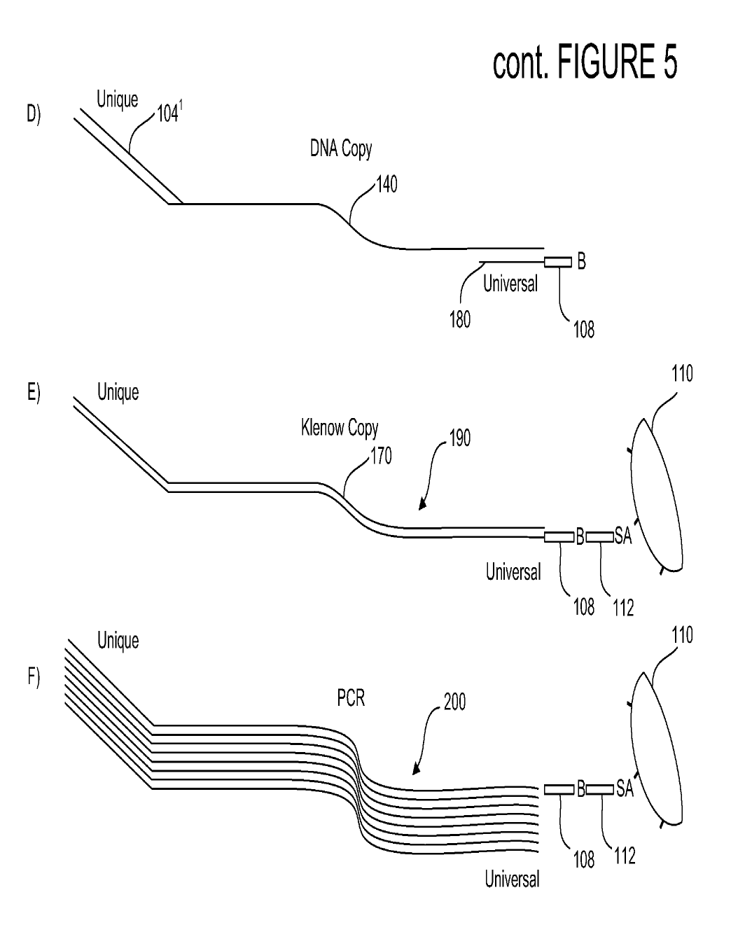

In accordance with another aspect, as shown in FIG. 5, the unique-universal probe 100 comprising universal sequence 102 and unique sequence 104 is not attached to a solid phase, but is instead allowed to function as a primer for the reverse transcriptase (RT)(Fig. 5a). RNA 130 in the sample hybridizes to the universal probe sequence 104 at a complimentary sequence 132 (FIG. 5b). The resulting hybrid 134 is used as the primer/template for reverse transcriptase (RT) to copy the hybridized bacterial RNA, thereby forming a cDNA extension strand 140 (FIG. 5c). After RT, a second strand 170 is made using Klenow DNA Polymerase and a 2^{nd} universal primer 180 that has been synthesized with a biotin 108 on its 5′ end (FIG. 5d). After the Klenow step, the double-stranded product 190 can now attach to a streptavidin molecule 112 on the solid phase substrate 110 (FIG. 5e). In this embodiment, the solid phase substrate 110 comprises magnetic beads. After washing, the solid phase substrate 110 with attached product 190 can be treated enzymatically or in any other way to remove any residual universal-unique probe 100 prior to PCR. PCR is then conducted to amplify the double-stranded product 190 to produce product 200 (FIG. 5f).

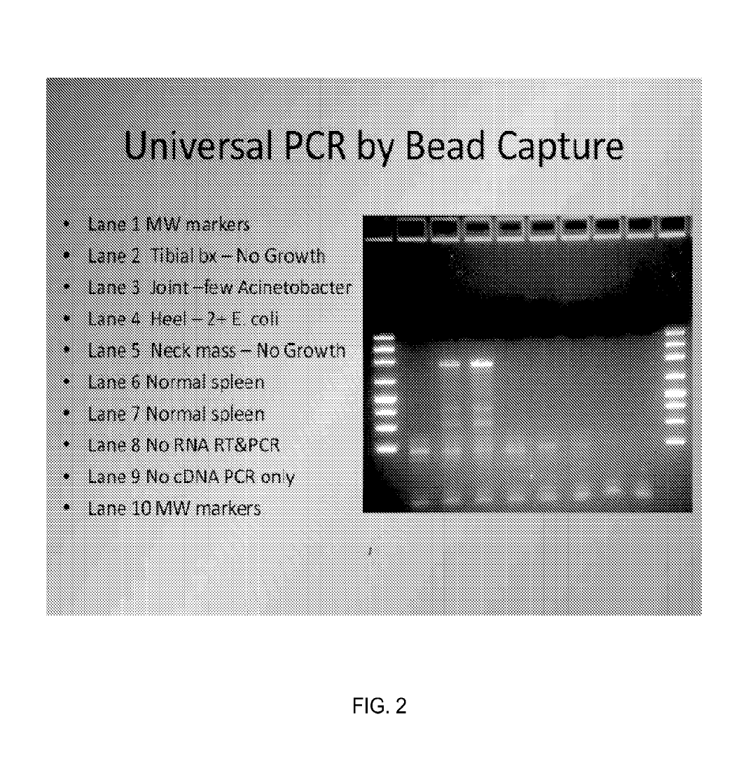

Referring to FIG. 2, FIG. 2 shows a gel where various samples were used to demonstrate the selectivity of the method embodiments. As shown, lanes 3 and 4, which were known to have bacterial infection, show a clear band of a specific molecular weight related to the bacterial PCR primer chosen illustrating the amplified target product. In addition, the inventors have discovered that commercially available reverse transcriptase is actually contaminated with nucleic acid sequences. Moreover, these contaminating sequences can interfere with the detection of nucleic acids according to the methods described herein. Accordingly, in a specific embodiment, reverse transcriptase is enzymatically treated prior to use to clean it of these contaminating sequences. Thus, one aspect of the invention pertains to nucleic acid-free reverse transcriptase. Enzymes used for this purpose include endonuclease(s). The cleaned reverse transcriptase, or nucleic acid-free reverse transcriptase, is then used in the process as described above. Any other enzymes used prior to the PCR, for example, Klenow reagent to make the reverse transcriptase cDNA product double-stranded, are likewise rendered non-contaminated.

In another aspect, the probe can be blocked upon capture of target nucleic acid material. This would be done after subjecting the probe to reverse transcriptase.

Nucleotides would typically be used to block the remaining probe not extended by reverse transcriptase. In a more specific embodiment, the nucleotides are deoxynucleotides. In a specific example, deoxythymidine triphosphate, or a similar deoxynucleotide is used to block the probe.

### EXAMPLES

### Example 1

After reverse transcription, the reaction mixture was diluted significantly (generally in the range of 1:20-1:60). Because the fusion primer not only can give a false positive from reagents alone, but can also interfere with the sensitivity of the PCR itself, the fusion primer was designed that the universal sequence portion is relatively short and the unique (non-bacterial) sequence is relatively long. By lengthening the universal part, the melting temperature (Tₘ) of the primer is reduced down to the range of 45-55° C. thus reducing its binding affinity. By lengthening the unique portion, the Tₘ is raised thus raising the temperature of the PCR to an annealing temperature of 65-70° C. as opposed to the standard 55-60° C. One of the critical aspects of the present invention is that the universal sequence does not hybridize with the contaminating DNA in the Taq enzyme during the PCR because the Tₘ of the universal sequence is more than 10° C. lower than the lowest temperature in the PCR. In order to ensure the second universal primer does hybridize, its Tₘ remained high by lengthening it without losing its universality characteristics. Modified nucleotides, for example, may be used that raise the Tₘ of a primer into which they have been incorporated Proof of principle was demonstrated of both the need and effectiveness of dilution, as well as the effectiveness of lowering the Tₘ of the universal portion of the fusion primer while raising the Tₘ of the unique portion and its corresponding 2^{nd} PCR primer.

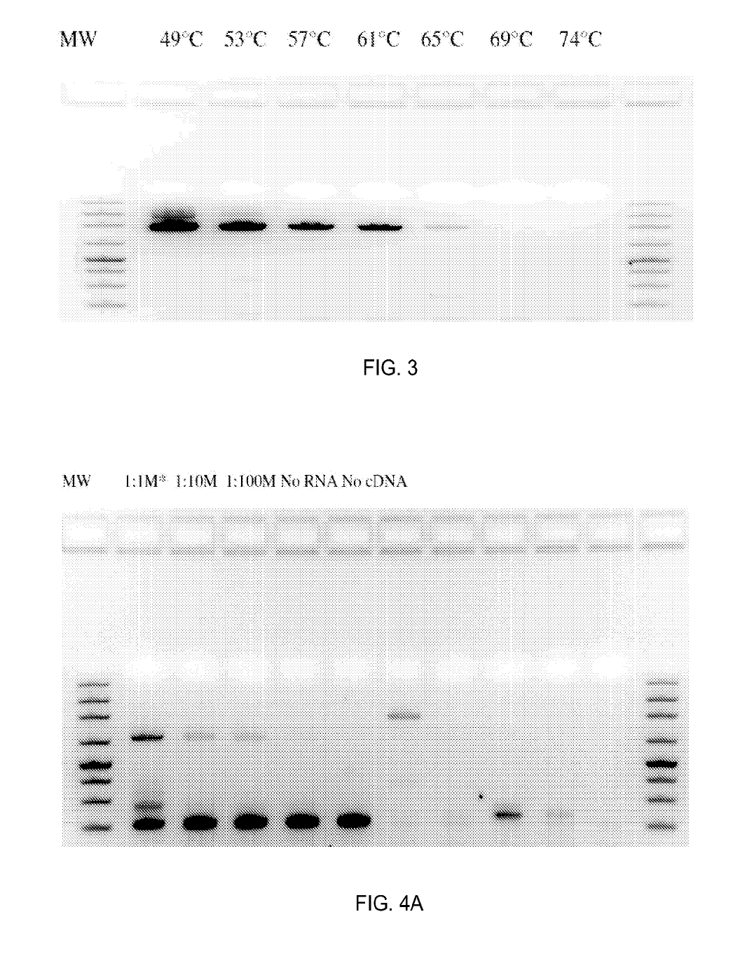

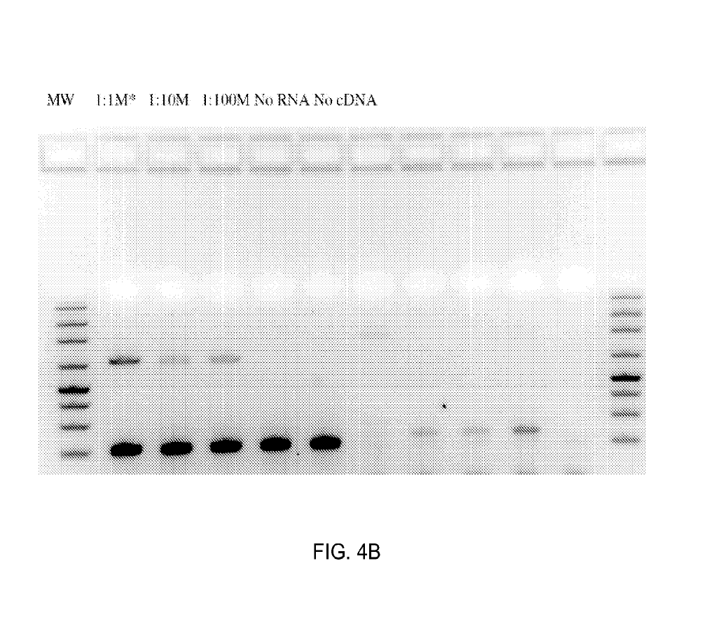

FIG. 3 shows the temperature dependence of the false positive product with reagents alone, namely that if the Tₘ of the universal part of the fusion primer is low enough, there can be no false positive PCR product if the PCR is carried out at a high enough temperature. FIGS. 4-5 show proof of principle that includes both dilution of the RT reaction mixture (1:50) and by using a high enough annealing temperature in the PCR. Both full sensitivity (in this case approximately 200 copies/reaction mixture) and no false positives were obtained at annealing temperatures of 65° C. and 68° C.

### Example 2

Pseudomonas aeruginosa RNA was diluted using a preparation that corresponds to approximately 200 copies/reaction mixture at 1:100M. The lanes that are labeled in FIGS. 3,-4a, and 4b correspond to an approximately 230 bp product while the unlabeled lanes are from a longer PCR product of around 450 bp. The reaction is significantly more sensitive when carrying out the shorter PCR. The reverse transcriptase reaction mixture was diluted 1:50 before performing the PCR.

It should be borne in mind that all patents, patent applications, patent publications, technical publications, scientific publications, and other references referenced herein and in the accompanying appendices are hereby incorporated by reference in this application to the extent not inconsistent with the teachings herein. It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those skilled in the art without materially departing from the invention herein.

For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

1. A probe for detecting target nucleic acid material in a sample, the probe comprising:
a universal probe sequence hybridizable to a target sequence;
a unique primer sequence interconnected to the probe sequence; and
a solid-phase medium associated with said probe sequence and unique primer sequence.

2. The probe of claim 1, wherein in the unique primer sequence is engineered to avoid binding with non-target nucleic acid material or contaminants in the sample.

3. The probe of claim 1, wherein the target nucleic acid material is a polynucleotide from an organism or virus.

4. The probe of claim 2, wherein the target nucleic acid material is bacterial, fungal, viral, or any other infectious agent.

5. The probe of claim 1, wherein the unique primer sequence is adjacent to the probe sequence.

6. The probe of claim 1 where in the unique primer sequence is engineered to avoid binding with non-target nucleic acid material or contaminants in the sample.

7. A probe for detecting bacterial nucleic acid material in a sample, the probe comprising:
a universal probe sequence hybridizable with polynucleotide sequences of multiple bacterial species; and
a non-bacterial primer sequence interconnected with said universal probe sequence.

8. The probe of claim 7, further comprising a solid-phase medium associated with said universal probe sequence and nonbacterial primer sequence.

9. The probe of claim 8, wherein said solid-phase medium is a bead.

10. The probe of claim 8, wherein said solid-phase medium is bead comprising streptavidin.

11. The probe of claim 8, wherein said solid-phase medium is a wall of a well, dish or other container capable of holding a fluid.

12. The probe of claim 7, wherein said universal probe sequence is an RNA or DNA sequence.

13. The probe of claim 7, wherein said universal probe sequence and said non-bacterial primer sequence are on the same strand.

14. The probe of claim 7, wherein said non-bacterial primer sequence comprises a sequence of at least 5, 10, 15, 20, or 25 bases that are lacking in 10 or more natural species of bacteria.

15. The probe of claim 8, wherein said probe further comprises a spacer sequence between the solid phase medium and the probe sequence or primer sequence.

16. The probe of claim 15, wherein the spacer sequence is a strand of common nucleic acid bases linked to the non-bacterial primer sequence on one end and to biotin on the other end, and wherein said biotin is bound to said bead.

17. The probe of claim 1, wherein said solid-phase medium is a magnetic bead.

18. The probe of claim 1, wherein said universal probe sequence is an RNA or DNA sequence specific to 16S RNA of multiple bacterial species.

19. The probe of claim 1, wherein said bacterial nucleic acid material comprises bacterial DNA or RNA sequences, or both.

20. A probe for detecting bacterial nucleic acid material in a sample, the probe comprising:
a probe strand comprising
(i) a universal probe sequence hybridizable with polynucleotide sequences of multiple bacterial species, the universal probe sequence having a Tm of from 45-55° C.; and
(ii) a non-bacterial primer sequence interconnected with said universal probe sequence.

21. The probe of claim 20, further comprising an adenine strand linked with the probe strand on one end and biotin on the other end and a solid-phase medium comprising streptavidin bound to said biotin.

22. A method of detecting bacterial nucleic acid material in a sample, the method comprising
contacting the sample with a probe comprising (i) a universal probe sequence hybridizable with polynucleotide sequences of multiple bacterial species; (ii) a nonbacterial primer sequence interconnected with said universal probe sequence; and
selectively amplifying any bacterial nucleic acid material in said sample that is captured by said probe.

23. The method of claim 22, wherein the probe further comprises (iii) a solid-phase medium associated with said universal probe sequence and said non-bacterial primer sequence.

24. The method of claim 22, wherein the bacterial nucleic acid material captured by the probe is a DNA or RNA sequence.

25. The method of claim 22, wherein the bacterial nucleic acid material captured by the probe is an RNA sequence.

26. The method of claim 22, further comprising subjecting the RNA sequence to reverse transcriptase under conditions to produce a DNA extension on the same strand as the universal probe sequence, the DNA extension being complementary to a portion of the RNA sequence not hybridized to the universal probe sequence.

27. The method of claim 26, wherein the universal probe sequence and DNA extension form a base strand, and wherein the method further comprises selectively amplifying comprises conducting a polymerase chain reaction (PCR) using said base strand.

28. The method of claim 27, wherein said PCR comprises real-time PCR.

29. The method of claim 27, wherein said selectively amplifying comprises conducting any known nucleic acid amplification method.

30. The method of claim 27, wherein said PCR includes traditional PCR.

31. The method of claim 27, wherein said PCR comprises combining said base strand, whether associated with said solid-phase medium or not, in a reaction mixture with a first primer complimentary to said non-bacterial primer sequence and a second primer complimentary to a sequence on said DNA extension.

32. A method of detecting target nucleic acid material in a sample, the method comprising
contacting the sample with a probe comprising a probe sequence hybridizable to a target sequence; a unique primer sequence interconnected to the probe sequence; and a solid-phase medium associated with said probe sequence and unique primer sequence; and
selectively amplifying any target nucleic acid material in said sample that is captured by said probe.

33. The method of claim 32, wherein the target nucleic acid material is an RNA sequence, and further comprising subjecting the captured RNA sequence to reverse transcriptase under conditions to produce a DNA extension on the same strand as the universal probe sequence, the DNA extension being complementary to a portion of the RNA sequence not hybridized to the universal probe sequence.

34. The method of claim 33, wherein the universal probe sequence and DNA extension form a base strand and said selectively amplifying comprises conducting a polymerase chain reaction (PCR) using said base strand.

35. The method of claim 32, further comprising blocking the probe after capture of the RNA sequence but prior to subjecting the RNA sequence to reverse transcriptase, and/or after subjecting the RNA sequence to reverse transcriptase.

36. The method of claim 35, wherein said blocking comprises contacting the probe with nucleotides.

37. The method of claim 35, wherein said blocking comprises contacting the probe with deoxynucleotides.

38. The method of claim 35, wherein said blocking comprises contacting the probe with deoxythymidine triphosphate.

39. The method of claim 35, wherein blocking occurs before subjecting the RNA sequence to reverse transcriptase.

40. The method of claim 35, wherein blocking occurs after subjecting the RNA sequence to reverse transcriptase.

41. Nucleic-acid free reverse transcriptase.

42. A method of detecting target nucleic acid material in a sample, the method comprising
contacting the sample with a fusion primer comprising: (a) a universal probe sequence hybridizable with polynucleotide sequences of multiple bacterial species; and (b) a non-bacterial sequence interconnected with said universal probe sequence;
subjecting the captured polynucleotide sequence to reverse transcriptase under conditions to produce a base strand having a DNA extension on the same strand as the universal probe sequence and the non-bacterial sequence;
conducting a polymerase chain reaction (PCR) using the base strand comprising the non-bacterial sequence, the universal probe sequence and the DNA extension; and
wherein the PCR comprises:
i) with the DNA strand dissolved in a PCR reaction mixture comprising said DNA strand, primers, a DNA polymerase, heating said PCR reaction mixture sufficiently to achieve denaturation of the base strand into single-strand DNA, wherein the primers comprise a first primer complimentary to said non-bacterial primer sequence and a second primer complimentary to a sequence on the DNA extension;
ii) cooling the PCR reaction mixture sufficiently to cause the primers to anneal to said single-strand DNA and to elongate and thereby at least partially form a DNA strand complementary to said single-strand DNA; and
iii) subjecting said PCR reaction mixture to a reaction temperature of about 65° C. to further elongate said complementary DNA strand formed in step (ii); and
iv) repeating steps (i)-(iii) to define a subsequent PCR step;
wherein the universal probe sequence is rendered at least partially inoperable to participate in the PCR at least before the subsequent PCR step.

43. The method of claim 42, wherein the universal probe sequence is inactivated during the subjecting step.

44. The method of claim 42, wherein the temperature differential between a Tₘ of the universal probe sequence and the reaction temperature in step (iii) is effective to preventive hybridization of universal probe sequence with contaminants in the PCR reaction mixture.

45. The method of claim 42, wherein the universal probe sequence has a Tₘ that at least 10 degrees lower than the reaction temperature.

46. The method of claim 42, wherein the universal probe sequence is engineered to bind to >90% of known bacterial isolates.

47. The method of claim 42, wherein the universal probe sequence is removed from the PCR reaction mixture so that it cannot participate in the subsequent PCR step.

48. The method of claim 42, wherein the universal probe sequence is constructed in such a manner that it cannot participate in the subsequent PCR step.

49. The method of claim 42, wherein the universal probe sequence is shortened to the extent that it cannot form a PCR product in the subsequent PCR step.

50. The method of claim 42, wherein the universal sequence is modified or contains modified nucleotides such that it cannot form a PCR product in the subsequent PCR step.

51. The method of claim 49, wherein the modified nucleotides are effective to increase the affinity of the universal sequence for RNA.

52. The method of claim 48, wherein the modified nucleotides are effective increase the affinity of either the non-bacterial sequence or the universal probe sequence such that the PCR can be carried out at sufficiently high temperature such that a PCR product comprising the universal probe sequence from the PCR reagents is not generated.

53. The method of claim 41, further comprising diluting the PCR reaction mixture prior to said heating in step (i) of the PCR.

54. The method of claim 41, wherein the PCR reaction is diluted with buffer in a range of 1:20 to 1:60 by volume.

55. The method of claim 41, wherein the fusion primer is enzymatically destroyed prior to the PCR.
